(19) United States
(12) Patent Application Publication
(10) Pub. No.: US 2024/0378773 A1
(43) Pub. Date: Nov. 14, 2024

(54) CREATING AN X-RAY RECONSTRUCTION WITH ULTRASOUND SUPPORT

(71) Applicant: Siemens Healthineers AG, Forchheim (DE)

(72) Inventor: Alois Regensburger, Poxdorf (DE)

(21) Appl. No.: 18/648,750

(22) Filed: Apr. 29, 2024

(30) Foreign Application Priority Data
May 8, 2023 (DE) 10 2023 204 237.1

(51) Int. Cl. G06T 11/00; A61B 6/00; A61B 6/03; A61B 6/40; G06T 7/30

(52) U.S. Cl. CPC G06T 11/008; A61B 6/032; A61B 6/4085; A61B 6/5247; G06T 7/30; G06T 2207/10081; G06T 2207/10136; G06T 2207/30004; G06T 2210/41; G06T 2211/464

(57) ABSTRACT

Reconstruction results of X-ray recordings are to be improved without any additional radiation burden. For this purpose, a method for creating an X-ray reconstruction by way of acquiring an X-ray recording of an object and creating a first reconstruction from the X-ray recording is provided. Within the first reconstruction, an uncertainty region is determined. In addition, an ultrasound recording restricted to a part of the object that corresponds to the uncertainty region is acquired. Further, the data of the first reconstruction is combined with data of the ultrasound recording to create a second reconstruction.

The present patent document claims the benefit of German Patent Application No. 10 2023 204 237.1, filed May 8, 2023, which is hereby incorporated by reference in its entirety.

### TECHNICAL FIELD

The present disclosure relates to a method for creating an X-ray reconstruction. In addition, the present disclosure relates to a corresponding X-ray apparatus as well as a computer program and an electronically readable data carrier.

### BACKGROUND

Modern X-ray examinations are based, for example, on the so-called DE-CBCT technique (Dual-Energy Cone-Beam Computed Tomography). This examination with different X-ray energies enables a more exact tissue differentiation than previously. The dose, however, corresponds to that of a conventional CT examination. The image reconstruction of such CBCT recordings is a challenging problem, in particular in the case of incomplete projection data (detector truncation). In this regard, a model-based reconstruction, such as ART (Algebraic Reconstruction Technique) has proved to be particularly promising. Therein, a three-dimensional (3D) model of the object is created and by way of iterative back projection and adjustment to the projection recordings, is refined act by act.

Such model-based methods for image reconstruction, which may also include dedicated acts for correcting artifacts such as truncation, function satisfactorily for a majority of patients. In some cases, the iterative image reconstruction runs into a local minimum or does not converge at all, which is normally equivalent to large errors in the 3D image reconstruction.

So far, there have hardly been any possibilities for “rescuing” an insufficiently reconstructable DE-CBCT recording retrospectively.

Where reconstruction problems arise, a renewed scan with changed parameters (for example, a large-volume recording, a higher recording dose, better breath holding) or even a CT recording in another space are currently necessary. This implies an additional dose burden and possibly contrast medium burden for the patient and additional time expenditure for the personnel.

Movement correction algorithms are known from Rohkohl et al., “Interventional 4-D Motion Estimation and Reconstruction of Cardiac Vasculature without Motion Periodicity Assumption,” Medical Image Computing and Computer-Assisted Intervention—MICCAI 2009, Vol. Lecture Notes in Computer Science, 5761 Heidelberg: Springer 2009, pp. 132-139-ISBN 978-3-642-04267-6.

### SUMMARY AND DESCRIPTION

The object of the present disclosure relates to improving X-ray reconstructions with a small data processing effort without an increased radiation burden.

The scope of the present disclosure is defined solely by the appended claims and is not affected to any degree by the statements within this summary. The present embodiments may obviate one or more of the drawbacks or limitations in the related art.

According to the disclosure, a method is thus provided for creating an X-ray reconstruction. The X-ray reconstruction creates a 2D or 3D image of an object to be examined (e.g., a patient), which may be acquired with the help of a model and/or a reconstruction algorithm from one or more X-ray recordings.

In the method therefore, an X-ray recording of an object is acquired. If relevant, a plurality of X-ray recordings are acquired. The X-ray recordings may be 2D or 3D recordings.

In a further act, a creation of a first reconstruction from the X-ray recording and/or the X-ray recordings takes place. The first reconstruction may take place with a known reconstruction algorithm. This is possibly an iterative reconstruction. The reconstruction algorithm for the first reconstruction provides, for example, a rough model, in particular a reconstruction model.

In a further act, an (e.g., automatic) determination of a geometry or a structure of an uncertainty region takes place within the first reconstruction. An uncertainty region means that the first reconstruction is uncertain in this region or is not present at all. For example, the first reconstruction is unsharp in a particular region due to movement, covering, or suchlike. The uncertainty region may however also arise by way of a truncation if, for example, only a part of the object is captured by the intended recording region of an X-ray device (e.g., target recording region and/or reconstruction volume). Regions at the edge of this target recording region or outside the target recording region may be uncertainty regions in which parts of the object are represented only with reduced quality or not at all.

The geometry, extent, or structure of the uncertainty region may be determined manually or automatically. If the uncertainty region is characterized, for example, by unsharpness, the automatic determination may take place based on edge gradients or suchlike. If, for example, truncations in CBCT recordings are the cause for the uncertainty region, the already known radiation geometry may be used for automatic determination of the uncertainty region. Otherwise if, for example, a vascular tree is represented unsharp and therefore an uncertainty region is defined, the geometry of the vascular tree and thus of the uncertainty region may be detected automatically by image recognition. The automatic determination of the uncertainty region lies within the first reconstruction and is therefore smaller than the first reconstruction region. Thus a partial region of the first reconstruction that does not extend beyond the entire first reconstruction is defined as an uncertainty region.

In a further act, an acquisition of an ultrasound recording is restricted to a part of the object corresponding to the uncertainty region. This means that the ultrasound recording may only take place in the uncertainty region. The part of the object may be smaller than the entire object in the beam space and/or the beam cone. However, the region of the ultrasound recording need not exactly correspond to the uncertainty region. However, the uncertainty region may cover more than half of the ultrasonically-examined part of the object.

Finally, a combining of data of the first reconstruction with data of the ultrasound recording to create a second reconstruction takes place. The combining of the data may include a replacement of data, an amendment of data and an addition. The ultrasound data is thus used to improve the first reconstruction in that a corresponding second reconstruction is created based on a refined model, in particular with a known reconstruction algorithm, for example, the same reconstruction algorithm for creating the first reconstruction. In particular, the second reconstruction may be created by an iterative reconstruction making use of the refined model. The ultrasound data is thus used for refining the reconstruction model only in the uncertainty region. Because only part of the reconstruction region is improved and/or optimized, the optimized reconstruction may take place quicker than in the case that the reconstruction model is refined in the entire reconstruction region.

In an embodiment, the X-ray recording is a CBCT recording. The X-ray recording is thus created by way of a cone beam. As a result, with relatively large objects, truncations may occur. With the reconstruction optimization by ultrasound, in particular in truncated regions, uncertainty regions in CBCT recordings may be reduced.

In a further embodiment, it is provided that the geometry of the uncertainty region relates to a location and/or an extent within the first reconstruction. The uncertainty region may relate, for example, to a point and/or a location with predetermined surroundings. For example, an uncertainty region may be defined by way of a mid-point that is then used as a mid-point for the ultrasound recording that in turn covers a predefined region. The geometry of the uncertainty region may however also relate to its extent, which is determined dynamically. For example, the extent of an unsharp region may be established by image processing algorithms that assess the sharpness. In this way, the uncertainty region may be established in a situation-related manner.

In a further embodiment, the ultrasound recording may be acquired by way of a technique based on shear waves, Doppler ultrasound, or image capture by higher harmonics. These techniques are suitable for emphasizing different tissues and structures better. In this way, it is possible using these techniques to undertake suitable tissue and/or structure classifications.

In a further embodiment, it may be provided that both the first reconstruction and also the ultrasound recording are 3D recordings. These two 3D recordings may then be registered to one another. By way of the combining of this 3D data, high quality volume recordings are obtained.

In a further embodiment, the data of the ultrasound recording includes material information, tissue information, or structural information. Information of this type may thus be integrated into the first reconstruction. In this way, tissue types may be better distinguished in the reconstruction. Thus, for example, bones, soft tissue, blood vessels, skin, and suchlike may be better separated from one another. This material information and/or structural information may however also be used to be able to determine the extent of organs, (e.g., the liver), more exactly.

According to a further embodiment, an ultrasound device is controlled for acquiring the ultrasound recording dependent upon the geometry of the uncertainty region. Thus, for example, the geometry information relating to the uncertainty region may be used to guide and/or steer an ultrasound device. In this way, it is possible to capture only the regions actually of interest with the ultrasound in order to refine the reconstruction. Alternatively to the controlling of the ultrasound device based on the data of the uncertainty region, a simple registration of the ultrasound recording with the first reconstruction based on the image data may take place.

In another embodiment, the uncertainty region is situated at least partially outside a target recording region of an X-ray device used for the X-ray recording. This is the case, for example, if the patient and/or the object is very large. In order nevertheless to be able to reconstruct organs situated outside the target recording region of the X-ray device, the ultrasound data may be utilized.

In a further embodiment, it is provided that the automatic determination of the geometry of the uncertainty region takes place based on an image contrast of the first reconstruction, an image sharpness of the first reconstruction or a convergence rate of an algorithm used for the first reconstruction. Thus, for example, the extent or the geometric form of the uncertainty region is established based on the image contrast of the first reconstruction. If the image contrast is, for example, below a predetermined threshold, this may be used as a criterion for the uncertainty region. Similarly, the image sharpness may also be assessed based on a predetermined threshold and the uncertainty region may be determined by this assessment. Alternatively, for specifying the uncertainty region, the convergence rate of the reconstruction algorithm may also be used. If the convergence rate in an image region is, for example, below a particular threshold value, then this partial region may be counted as being within the uncertainty region. The recognition of image artifacts or metal objects or particular other objects or anatomical structures known to lead to image artifacts in their surroundings may serve as a further example for determining the uncertainty region. Thus, the uncertainty region may be determined automatically in a variety of ways. Alternatively, the uncertainty region may naturally also be specified manually, for example, by marking it with a pen.

In a further embodiment, a movement correction of at least a part of the first reconstruction is carried out with the ultrasound data to acquire the second reconstruction. This may be carried out when movements due to breathing or the heartbeat cause the regions to be examined to appear unsharp. For the movement correction, known movement correction algorithms such as CAVAREC may be used. If this movement correction based solely on the first reconstruction fails, ultrasound data may additionally be utilized in order to compensate for any movements in partial regions of the first reconstruction. Thus, the correction scope with regard to movements may be significantly widened.

In a further embodiment, the combining of the data of the first reconstruction with data of the ultrasound recording includes a registration of the ultrasound recording to the first reconstruction. As already indicated above, 2D or 3D ultrasound recordings may accordingly be registered to parts of the first reconstruction. By way of the combining of the data, the reconstruction model is refined accordingly.

Furthermore, in an embodiment, a specifiable high contrast object may be determined as the structure, a position of the high contrast object may be acquired by way of triangulation, and the registration of the ultrasound recording to the first reconstruction may take place dependent upon the position. The possibly predefined high contrast object may be an artificial object or a known object, (e.g. a large vessel), which stands out from the other objects due to high contrast. Due to the high contrast, the position of this high contrast object may be acquired reliably by way of triangulation, wherein the high contrast object may be recorded from different perspectives. From these different recordings, the position may be determined reliably.

The aforementioned object is also achieved by way of: an X-ray apparatus for creating an X-ray reconstruction with an X-ray unit configured for acquiring an X-ray recording of an object; a reconstruction unit (image processing, processor, memory store) configured for creating a first reconstruction from the X-ray recording; a determining unit configured for (automatically or manually) determining a geometry or a structure of an uncertainty region within the first reconstruction; an ultrasound unit configured for acquiring an ultrasound recording restricted to a part of the object corresponding to the uncertainty region; and a data processing unit configured for combining data of the first reconstruction with data of the ultrasound recording to create a second reconstruction.

The X-ray unit may include a C-arm device or a CT scanner. The reconstruction unit may be an image processing unit with a processor and a memory store. The determining unit for determining the geometry or the structure of the uncertainty region may include an image processing unit for automatic determination of the uncertainty region. Alternatively, the determining unit may also have an input interface with which an uncertainty region may be input manually, for example, drawn in. The ultrasound unit may be based upon different technologies such as shear wave technology, Doppler technology, etc. Furthermore, the ultrasound unit may be moved and/or controlled, if appropriate, by way of a control facility of the X-ray apparatus, in order to record the uncertainty region as precisely as possible within its limits. The data processing unit for combining and/or integrating the data of the first reconstruction and of the ultrasound recording may also possess image processing functionality and may be constructed with a processor and possible storage elements.

A computer program is also provided that may be loaded directly into a memory store of a control facility of the X-ray apparatus that is described above. The computer program has program means in order to carry out the acts of the aforementioned method when the program is executed in the control facility.

In a similar manner, an electronically readable data carrier with electronically readable control information stored thereon is also provided, wherein the control information includes at least the computer program as described above and being configured such that, on use of the data carrier in the control facility, it causes the X-ray apparatus to carry out a method as described above.

For application cases or application situations that may arise with the method and which are not explicitly described here, according to the method, an error message and/or a request for input of a user feedback is output and/or a standard setting and/or a predetermined initial state is set.

### BRIEF DESCRIPTION OF THE DRAWINGS

The present disclosure is now described in greater detail making reference to the accompanying drawings, in which:

FIG. 1 shows a schematic representation of an example of a capture of projection recordings of an examination object by way of a CT apparatus.

FIG. 2 shows a schematic representation of an example of an X-ray apparatus with an ultrasound unit.

FIG. 3 shows a schematic representation of an example of a sequence of a method.

### DETAILED DESCRIPTION

In a specific example, the present disclosure may be used for cone-beam computed tomography (CBCT) that involves an imaging method for generating a reconstruction mapping of an examination object. In cone-beam computed tomography, X-ray radiation is emitted by an X-ray radiation source to the examination object, wherein a main beam region opens out conically from the X-ray radiation source. Opposing the X-ray radiation source is a detector that detects the emitted X-ray beams as a two-dimensional projection recording of the examination object. The examination object is arranged between the X-ray radiation source and the detector. To enable the reconstruction of a reconstruction mapping of the examination object, a dataset including a plurality of projection recordings of the examination object is captured.

In order to capture the projection recordings of the examination object for the dataset, the X-ray radiation source and the detector are moved along a trajectory, (e.g., a round trajectory), about the examination object. The respective projection recordings of the examination object are created in respective positions along the trajectory. The dataset includes a plurality of projection recordings that map the examination object in different orientations. The projection recordings of the dataset are processed according to a predetermined reconstruction method in order to generate a reconstruction mapping of the examination object.

For an optimum reconstruction of the examination object, the examination object is penetrated by the X-ray beams in each of the positions. In other words, during the capture of each projection recording, the entire examination object is situated within the conical main beam region.

The case may exist that, during the capture of at least some of the projection recordings, out-of-limit regions of the examination object are situated outside the conical main beam region, and these are therefore not penetrated by the X-ray radiation. As a result, these out-of-limit regions of the examination object are also not mapped in the respective projection recording. Due to the lack of these out-of-limit regions of the examination object in some of the projection recordings, during the reconstruction of the reconstruction mapping of the examination object, errors may arise. This is attributable to the fact that a section passed through by X-ray beams is wrongly evaluated in a reconstruction model, so that the attenuation of the corresponding X-ray beams by the out-of-limit regions in question is not taken into account.

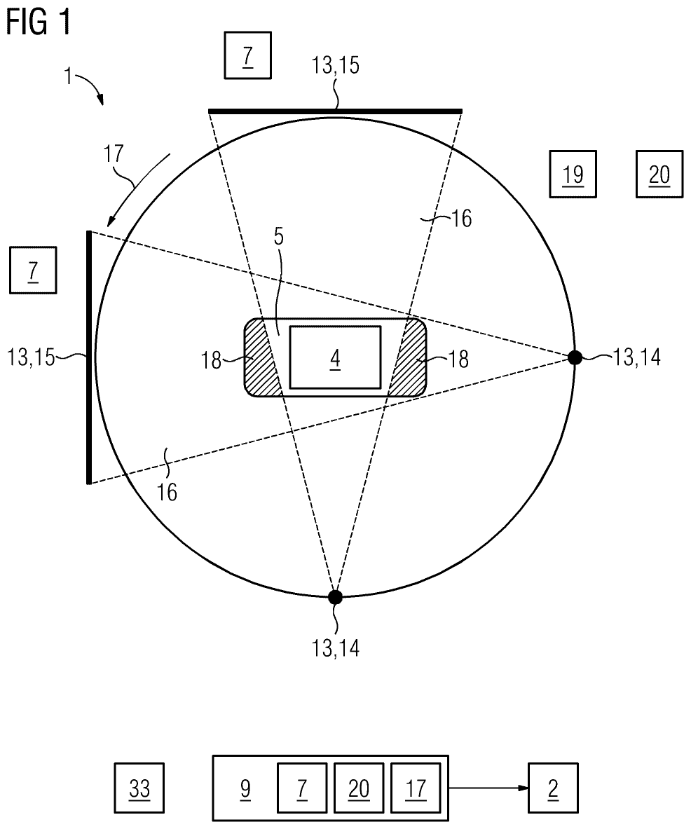

FIG. 1 shows a schematic representation of a specific capture of projection recordings 7 of an examination object 5 by way of a CT apparatus 1.

The CT apparatus 1 may be configured for carrying out a cone-beam computed tomography method in order to be able to generate a reconstruction mapping 2 (for short: reconstruction and/or first reconstruction) of an examination object 5 (for short: object). The CT apparatus 1 may have a reconstruction unit 33 that may be configured to acquire the reconstruction mapping 2 of the examination object 5 from projection recordings 7 of a dataset 9.

The CT apparatus 1 may have an X-ray unit 13 that may include an X-ray radiation source 14 and a capture screen 15. The X-ray radiation source 14 may be configured to emit X-ray radiation along a conical volume in the direction of the capture screen 15. The examination object 5 may be arranged between the X-ray radiation source 14 and the capture screen 15. The X-ray beams are absorbed by the examination object 5 so that the capture screen 15 captures a two-dimensional projection recording of the examination object 5. A reconstruction of the reconstruction mapping 2 of the examination object 5 may require a recording of a large number of projection recordings 7 of the examination object 5 from different directions. For this purpose, the CT apparatus 1 may be configured to move the X-ray unit 13 along a trajectory 17, (e.g., a circular trajectory), about the examination object 5. In predetermined directions, the respective projection recordings 7 of the examination object 5 are captured and added to the dataset 9. The reconstruction unit 33 may be configured to reconstruct the reconstruction mapping 2 of the first dataset 9 from the first projection recordings 7 of the first dataset 9 after a predetermined reconstruction method.

During each recording of the respective first projection recordings 7, the examination object 5 may be situated within the cone 16 so that the examination object 5 is completely transirradiated and the projection recording includes the examination object 5 over a whole respective dimension thereof. However, it may be the case that at least in some positions of the capturing facility 13, out-of-limit regions 18 of the examination object 5 are situated outside the cone 16 and are therefore not captured by way of the respective first projection recording. In this case, a so-called truncation takes place. As a result, errors may occur in the reconstruction mapping 2 of the examination object 5.

The CT apparatus 1 may have a sensor apparatus 19 that may be configured to capture a position of the examination object 5 or of an ultrasound unit. The sensor may include a camera and a position detector. The sensor data 20 may be added to the dataset 9 by way of the sensor apparatus 19.

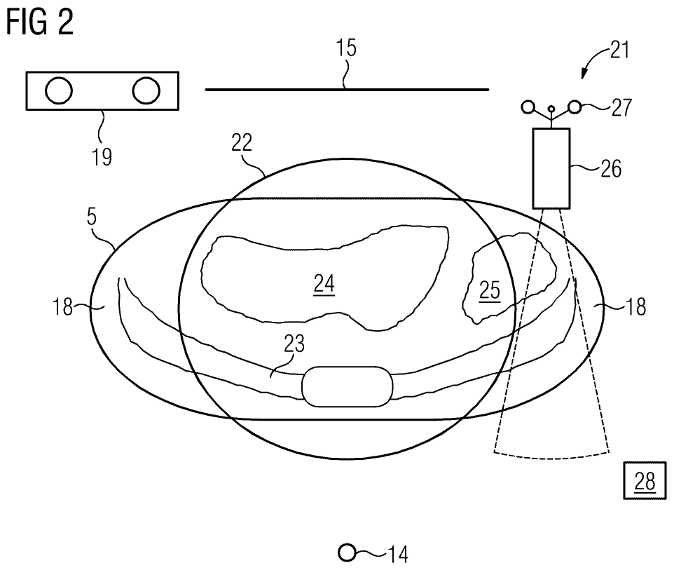

FIG. 2 shows schematically an embodiment of an X-ray apparatus with an additional ultrasound unit 21. The X-ray apparatus has an X-ray unit 13 with an X-ray radiation source 14 and an X-ray detector and/or a capture screen 15. There may be a target recording region 22 that may also be designated as the reconstruction volume. An examination object 5, (e.g. a patient), is situated here partially in the target recording region 22. The patient has bones 23, a first organ 24, and a second organ 25. Although the first organ 24 is situated within the target recording region 22 and/or the reconstruction volume, the second organ 25 is situated partially outside thereof. For this reason, the second organ 25 cannot be reconstructed exactly with the X-ray recordings alone.

The ultrasound unit 21 is provided in order to be able to provide additional data from the examination object 5 from inside and/or outside the reconstruction volume 22. It may have an ultrasound probe 26 and one or more tracking elements 27. For example, the tracking element reflectors may be for an optical tracking. In the example of FIG. 2, a truncated region (out-of-limit region 18) outside the reconstruction volume 22 is recorded with the ultrasound unit 21. By this, an ultrasound recording 28 may be acquired. This may be integrated into the dataset 9 for the reconstruction.

The optical tracking of the ultrasound unit 21 may take place with the aid of a sensor apparatus 19, in particular a camera. With this sensor apparatus, position information regarding the ultrasound unit may be established. This position information may itself be utilized for controlling the ultrasound unit 21 with the aid of a control facility (not shown).

In particular, the X-ray unit 13 may enable 3D recordings. Specifically, the X-ray unit 13 may be configured as a C-arm device for recording a CBCT.

Apart from the camera for the controlling and/or following of the ultrasound unit 21, another navigation system and/or tracking system registered to the coordinates of the X-ray system may be used. In particular, this may also be realized on an electromagnetic basis. Thereby, in particular, the position and the orientation of the ultrasound probe 26 (for 2D or 3D) may be captured.

In addition, the reconstruction unit 33 drawn in FIG. 1 may herein be present as a data processing unit. It may be equipped with a processor and a data store. The functionalities of a determining unit configured for determining a geometry or structure of an uncertainty region and also a data processing unit configured for combining data of the first reconstruction with data of the ultrasound recording to create a second reconstruction may also be integrated into the reconstruction unit 33.

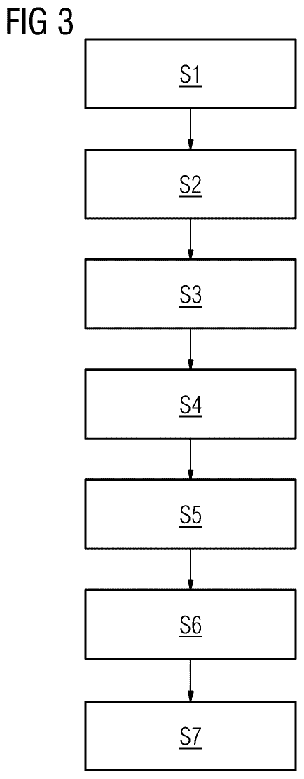

Suitable method acts for a first embodiment are now described with reference to FIG. 3.

In act S1, an X-ray recording is acquired. For example, this is a CBCT recording and/or a DE-CBCT recording.

In act S2, a first reconstruction from the X-ray recording and/or X-ray recordings may take place. This reconstruction may take place with a known method, in particular model-based and possibly iteratively.

In act S3, the determination of a geometry or a structure of an uncertainty region takes place within the first reconstruction. For example, the image regions in which the geometric reconstruction and/or the model-based material estimation has a large degree of uncertainty (“uncertainty regions”) is output by the reconstruction algorithm. These may be image regions in which the model does not converge, or only slowly converges, or image regions in which only insufficient information from the projection data is available for the model formation. Uncertain data may also be image information from the projection data that indicates, in the comparison thereof, that a truncation model is false or too inexact.

In act S4, a possibly registered and/or executed recording of ultrasound images may take place that covers at least a part of the uncertainty regions established in act S3. In any event, it does not cover the entire target recording region and/or the entire reconstruction volume 22 of the X-ray unit 13. The ultrasound images acquired serve for the recording of, for example, geometric information and, in particular, for tissue/structure classification, (e.g., by shear waves, Doppler ultrasound, image capture by higher harmonics, and so forth). Optionally, a 3D ultrasound image reconstruction may also be created.

In act S5, a registration of the ultrasound information into the first reconstructed volume takes place. This may take place via image information and/or tracking of the ultrasound head 26.

In act S6, the derivation of material properties, tissue properties, and/or geometric properties from the added ultrasound data may possibly take place. Corresponding material information, geometrical information, and other information may be added to the (spectral/tissue-resolved) reconstruction model, whereby a refined model results.

In act S7, a renewed (iterative) reconstruction (second reconstruction) takes place based on the refined model.

It is now described, based on a second specific embodiment, how the partial ultrasound support may advantageously be utilized in X-ray diagnostics and also for movement correction. Under some circumstances, CBCT recordings are acquired during organ movements. Under some circumstances, however, the patient is also very large and extends beyond the reconstruction volume. An initial reconstruction and/or first reconstruction may therefore be very unsharp, so that for example, a CAVARC approach, that is, the registration of projection images to a first vessel reconstruction, fails.

In this case also, a first reconstruction may take place. It may be marked, for example, subsequently by way of algorithmic (automatic) evaluation or by user input, as impaired too much by patient movements during the recording. As criteria for automatic evaluation, i.e., for the determination of uncertainty regions, the sharpness of reconstructed high contrast structures, other 3D image quality measures, feedback of the model-based reconstruction algorithm regarding existing uncertainties, and/or slow/lacking convergence may be utilized. Optionally, a first use of a known movement correction algorithm such as CAVAREC and a renewed evaluation may take place.

Optionally, a triangulation of the approximate position of large vessels and/or other high contrast objects from a plurality of projection images may take place. It is possibly checked whether these vessels/objects are reconstructed in the first reconstruction in sufficient quality and image sharpness. Alternatively or additionally, an identification of particularly unsharp and/or movement-impaired image regions may take place in the initial 3D image reconstruction.

Subsequently, a demand for the acquisition of at least one ultrasound image and/or 2D/3D ultrasound sweeps as the initial template for the movement-compensated reconstruction (including vessel segmentation from ultrasound) may take place in these image regions. Optionally, an at least partial 3D reconstruction of the ultrasound images may be carried out.

Thereafter, a matching and/or an image registration of the large vessels and/or the other high contrast objects identifiable in 2D projections to the corresponding structures in the ultrasound images and/or ultrasound reconstructions may take place.

Finally, a (CBCT) image reconstruction (second reconstruction) may be carried out based on the 2D projection images that were movement-compensated in the previous act.

Optionally, an iterative refinement similar to the CAVAREC algorithm may be implemented, if appropriate, with additional use of the acquired ultrasound data. The ultrasound data therefore does not have to be used in every refinement act.

In an advantageous manner, the present disclosure may thus contribute to the number of CBCT recordings that are made under problematic circumstances but which nevertheless may be used diagnostically and/or for therapy management being increased. A further advantage of the partial ultrasound support is that no additional dosage burden on the patient is required. In an advantageous realization, a C-arm imaging device with an integrated ultrasound unit that is navigable according to the determined uncertainty region is provided.

Overall, the disclosure has the invaluable advantage that lacking/unsharp information may be subsequently captured in order to achieve a subsequent improvement in the reconstruction result.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for creating an X-ray reconstruction, the method comprising:
acquiring an X-ray recording of an object;
creating a first reconstruction from the X-ray recording;
determining a geometry or a structure of an uncertainty region within the first reconstruction;
acquiring an ultrasound recording restricted to a part of the object corresponding to the uncertainty region; and
combining data of the first reconstruction with data of the ultrasound recording to create a second reconstruction.

2. The method of claim 1, wherein the X-ray recording is a Cone-Beam Computed Tomography (CBCT) recording.

3. The method of claim 1, wherein the geometry of the uncertainty region relates to a location and/or an extent within the first reconstruction.

4. The method of claim 1, wherein the ultrasound recording is acquired by way of a technique based on shear waves, Doppler ultrasound, or image capture by higher harmonics.

5. The method of claim 1, wherein both the first reconstruction and the ultrasound recording are three-dimensional (3D) recordings.

6. The method of claim 1, wherein the data of the ultrasound recording comprises material information, tissue information, or structural information.

7. The method of claim 1, wherein an ultrasound unit is controlled for acquiring the ultrasound recording in dependence upon the geometry of the uncertainty region.

8. The method of claim 1, wherein the uncertainty region is situated at least partially outside a target recording region of an X-ray device used for the X-ray recording.

9. The method of claim 1, wherein the determining of the geometry of the uncertainty region takes place based on an image contrast of the first reconstruction, an image sharpness of the first reconstruction, or a convergence rate of an algorithm used for the first reconstruction.

10. The method of claim 1, wherein a movement correction of at least a part of the first reconstruction is carried out with the ultrasound data to acquire the second reconstruction.

11. The method of claim 1, wherein the combining of the data of the first reconstruction with data of the ultrasound recording comprises a registration of the ultrasound recording to the first reconstruction.

12. The method of claim 11, wherein a specifiable high contrast object is determined as the structure,
wherein a position of the high contrast object is acquired by way of triangulation, and
wherein the registration of the ultrasound recording to the first reconstruction takes place in dependence upon the position of the high contrast object.

13. An X-ray apparatus for creating an X-ray reconstruction, the X-ray apparatus comprising:
an X-ray unit configured to acquire an X-ray recording of an object;
a reconstruction unit configured to create a first reconstruction from the X-ray recording;
a determining unit configured to determine a geometry or a structure of an uncertainty region within the first reconstruction;
an ultrasound unit configured to acquire an ultrasound recording restricted to a part of the object that corresponds to the uncertainty region; and
a data processing unit configured to combine data of the first reconstruction with data of the ultrasound recording to create a second reconstruction.

14. An electronically readable data carrier with electronically readable control information stored thereon that comprises a computer program and is configured such that, when the data carrier is used in a control facility, an X-ray apparatus is triggered to:
acquire an X-ray recording of an object;
create a first reconstruction from the X-ray recording;
determine a geometry or a structure of an uncertainty region within the first reconstruction;
acquire an ultrasound recording restricted to a part of the object corresponding to the uncertainty region; and
combine data of the first reconstruction with data of the ultrasound recording to create a second reconstruction.
